(19) United States
(12) Patent Application Publication
(10) Pub. No.: US 2025/0088812 A1
(43) Pub. Date: Mar. 13, 2025

(54) FITTING AGENT FOR A HEARING DEVICE WITH ENVIRONMENT MODEL

(71) Applicant: GN Hearing A/S, Ballerup (DK)

(72) Inventors: Tanya IGNATENKO, Eindhoven (NL); Kirill KONDRASHOV, Eindhoven (NL); Aalbert DE VRIES, Eindhoven (NL)

(73) Assignee: GN Hearing A/S, Ballerup (DK)

(21) Appl. No.: 18/793,727

(22) Filed: Aug. 2, 2024

(30) Foreign Application Priority Data
Sep. 7, 2023 (EP) 23196116.0
Apr. 8, 2024 (EP) 24169028.8

(51) Int. Cl. H04R 25/00

(52) U.S. Cl. CPC H04R 25/70; H04R 25/505; H04R 2225/41

(57) ABSTRACT

A fitting agent for a hearing device system comprising a hearing device includes one or more processors configured to initialize a user model and an environment model, the user model comprising a plurality of user preference functions and associated user response distribution; obtain environment data; determine a first initial environment probability of a first environment and a second initial environment probability of a second environment based on the environment data and the environment model, obtain a test setting comprising a primary test setting and a secondary test setting for the hearing device based on the first initial environment probability and the second initial environment probability; provide the test setting; obtain a user input of a preferred test setting indicative of a preference for either the primary test setting or the secondary test setting; and update the user model based on the preferred test setting and the environment data.

### RELATED APPLICATION DATA

This application claims priority to, and the benefit of, European Patent Application No. 23196116.0 filed on Sep. 7, 2023, and European Patent Application No. 24169028.8 filed on Apr. 8, 2024. The entire disclosures of the above applications are expressly incorporated by reference herein.

### FIELD

The present disclosure relates to hearing devices and related tools, methods, and systems in particular for one or more of determining, tuning, fitting and optimizing hearing device parameters. Thus, a fitting agent for a hearing device and related methods, in particular a method for updating a user model, are provided.

### BACKGROUND

Fitting and tuning of hearing devices or hearing aids has always been considered a tedious task of healthcare professionals (HCPs). Traditional approaches for fitting hearing device parameters rely on compensation of a user's hearing loss, based on audiograms, by applying rules such as NAL-NL1 or NAL-NL2. These rules, however, do not take into account specific user preferences.

Recent approaches involve preference learning for hearing devices.

EP 3 493 555 A1 relates to a method for tuning hearing device parameters of a hearing device and a hearing device. The method comprises initializing a model; obtaining an initial test setting defined by one or more initial test hearing device parameters; assigning the initial test setting as a primary test setting; obtaining a secondary test setting based on the model; outputting a primary test signal according to the primary test setting; outputting a secondary test signal according to the secondary test setting; detecting a user input of a preferred test setting; updating the model based on the primary test setting, the secondary test setting, and the preferred test setting; and in accordance with a determination that a tuning criterion is satisfied, updating the hearing device parameters of the hearing device based on hearing device parameters of the preferred test setting.

### SUMMARY

Challenges still remain in improving the tools, methods and devices allowing an improved fitting and tuning of hearing device parameters.

A fitting agent for a hearing device system comprising a hearing device worn by a hearing device user is disclosed, wherein the fitting agent comprises one or more processors configured to initialize a user model and an environment model, the user model comprising a plurality of user preference functions and associated user response distribution; obtain environment data indicative of a present environment; optionally determine a first initial environment probability of a first environment and/or a second initial environment probability of a second environment based on the environment data and/or the environment model, obtain a test setting comprising a primary test setting and a secondary test setting for the hearing device based on the first initial environment probability and/or the second initial environment probability; present the test setting to the hearing device user; obtain a user input of a preferred test setting indicative of a preference for either the primary test setting or the secondary test setting; and optionally update the user model for provision of an updated user model based on the preferred test setting and the environment data.

The present disclosure takes into account any differences in user preference in different environments and provides effective and memory-efficient user preference model update and/or effective and memory-efficient environment model update. In particular, the need for storing historical and specific user feedback (previous primary and secondary test settings and preference thereof) may be eliminated in turn reducing the memory requirements.

Further, environment-dependent user preference functions may provide simpler user preference functions in turn allowing a simple and effective way to configure one or more hearing device parameters of a hearing device. Further, the present disclosure provides an improved listening experience to the user by improving the modelling of the users preferred hearing parameter settings in different environments in turn resulting in optimized settings being applied in the hearing device, which in turn allows for an improved user experience.

Further, the present disclosure provides an efficient automated search for optimal hearing device parameters by incorporating a personalized environment model into the learning cycle. A fitting agent, devices, and methods are provided, that allows to learn environments in an efficient and minimally obtrusive way by empowering the user to take direct decisions and have direct impact on the fitting and/or tuning process.

Further, the present disclosure allows that hearing device parameters can be configured, such as fitted and/or tuned, during a normal operating situation and/or with a small number of user inputs/interactions. Thus, a simple and smooth user experience of the hearing device is provided.

### BRIEF DESCRIPTION OF THE DRAWINGS

The above and other features and advantages will become readily apparent to those skilled in the art by the following detailed description of exemplary embodiments thereof with reference to the attached drawings, in which:

FIG. 1 shows a schematic overview of the fitting agent with the context/environment-aware preference learning system,

FIG. 2 schematically illustrates a hearing system according to the present disclosure,

FIG. 3 illustrates interaction between a user and a fitting agent, and

FIG. 4 is a flow diagram of an exemplary method according to the present disclosure.

### DETAILED DESCRIPTION

Various exemplary embodiments and details are described hereinafter, with reference to the figures when relevant. It should be noted that elements of similar structures or functions are represented by like reference numerals throughout the figures. It should also be noted that the figures are only intended to facilitate the description of the embodiments. They are not intended as an exhaustive description of the invention or as a limitation on the scope of the invention. In addition, an illustrated embodiment needs not have all the aspects or advantages shown. An aspect or an advantage described in conjunction with a particular embodiment is not necessarily limited to that embodiment and can be practiced in any other embodiments even if not so illustrated, or if not so explicitly described.

A fitting agent is disclosed. The fitting agent or at least a first part thereof may be implemented, e.g. as an application, in an accessory device, such as an electronic device. The accessory device comprises an interface, a processor, and a memory. The accessory device may for example be or comprise a mobile phone, such as a smartphone, a smart-watch, a special purpose device, a computer, such as a laptop computer or PC, or a tablet computer. The fitting agent or at least a second part thereof may be implemented in a server device. The fitting agent or at least a third part thereof may be implemented in a hearing device.

The present disclosure relates to a hearing device system, fitting agent, accessory device and hearing device of the hearing device system, and related methods. The accessory device forms an accessory device to the hearing device. The accessory device is typically paired or wirelessly coupled to the hearing device. The hearing device may be a hearing aid, e.g. of the behind-the-ear (BTE) type, in-the-ear (ITE) type, in-the-canal (ITC) type, receiver-in-canal (RIC) type, receiver-in-the-ear (RITE) type, or microphone-and-receiver-in-the-ear (MaRIE) type. The hearing device may be a hearable, such as a pair of earbuds or a headset. Typically, the hearing device system is in possession of and controlled by the hearing device user.

The hearing device system may comprise a server device and/or a fitting device. The fitting device is controlled by a dispenser and is configured to determine configuration data, such as fitting parameters. The server device may be controlled by the hearing device manufacturer.

The fitting agent may be a fitting agent for a hearing device system comprising a hearing device worn by a hearing device user.

The fitting agent comprises one or more processors. The one or more processors are configured to initialize a user model and an environment model. The environment model is also denoted environmental model.

The user model comprises a plurality of user preference functions and associated user response distribution. The user response distribution is optionally state-dependent and can be formulated as a single distribution, where depending on the state the distribution it has a respective state-dependent form.

Each user preference function and environment distribution function of environment model have a probabilistic mapping between them.

The user model optionally represents one or a plurality of probabilistic descriptions of user responses, when comparing two sets of hearing device parameter settings. Integral parts of the user model include one or more, such as a plurality of user preference functions and one or more, such as a plurality of distributions of the user responses, also denoted user response distributions, to the presented choices of parameters. The user model comprises one or more distributions of parameters of the respective user preference functions. In other words, each user preference function has an associated distribution of hearing device parameters. In other words, the user model may include a first user preference function and associated first user response distribution, wherein the first user preference function is optionally associated with a first environment. The user model may include a second user preference function and associated second user response distribution, wherein the second user preference function is optionally associated with a second environment. The user model may include a third user preference function and associated third user response distribution, wherein the third user preference function is optionally associated with a third environment. The user model may include K user preference functions and associated user response distribution, wherein the k′th user preference function, k=1, 2, . . . , K is optionally associated with a k′th environment.

A vector of hearing device parameters is optionally defined on an M-dimensional continuous compact surface. In particular, hearing device parameters x are optionally defined on an M-dimensional hyper-cube, i.e., x∈[0,1]^{M}. In one or more exemplary fitting agents/methods, the hearing device parameters may be normalized by their physical range. The fitting agent/method, is configured to find optimized/improved values of hearing device parameters, also denoted θ for a particular user. The number M of hearing device parameters may be 1 and/or less than 100, such as in the range from 10 to 50. The number M of hearing device parameters may be larger than 20, such as in the range from 25 to 75.

In general, the user preference functions are unknown. The user preference functions ƒ(x, θ, Λ, k)=ƒ(x; θₖ, Λₖ), k=1, 2, . . . , K, where K is the number of user preference functions, may be parametric functions of hearing device parameters x∈[0,1]^{M}, e.g. with known form but unknown shape. This shape is optionally characterized by fitting or tuning parameters, θₖ∈[0,1]^{M} and a scaling matrix Λₖ. The scaling matrix Λₖ may be a positive-definite scaling matrix Λₖ. The scaling matrix may be a diagonal matrix Λₖ=diag ([λ₁, λ₂, . . . , λ_{M})]), λₘ∈ [symbol] ,m=1,2, . . . , M.

The user preference functions or at least one or more of the user preference functions of the K user preference functions may be unimodal.

In one or more exemplary fitting agents/method, distribution priors may be applied to each element of Λₖ. For example, Lognormal distribution priors may be applied to each element of Λₖ, e.g., log(λₘ)˜ [symbol] (μₘ,σₘ²) or Gamma distribution priors may be applied to each element of Λ, e.g., λₘ˜Gamma(αₘ, βₘ).

The scaling matrix Λₖ does not need to be a diagonal matrix. The scaling matrix Λₖ may be selected as Λₖ=L′*L, where L is a low-triangular matrix (also known as the Cholesky decomposition of Λₖ). Gaussian priors may be applied on each of the elements of L, e.g., Lᵢⱼ˜ [symbol] (μᵢⱼ,σᵢⱼ²).

The maximizing argument θₖ of a k′th environment may be a transformation of another variable, e.g. may be constrained by the following prior assumptions

$${θ}_{k} = Φ ( \hat{Y} ) , \qquad ( 2 )$$

where Φ(Ŷ) is a cumulative density function of a probability distribution, such as standard normal distribution, and f is a sample from another probability distribution. In one or more example fitting agent/methods, the maximizing argument θₖ may be constrained by the following assumptions on representing θₖ as a transformation of Ŷ and corresponding prior distribution of the transformed variable:

$${θ}_{k} = Φ ( \hat{Y} ) , with \hat{Y} ∼ ( {μ}_{k} , {Σ}_{k} ) , \qquad ( 3 )$$

where Φ(Ŷ)=∫_{-∞}^{Ŷ} [symbol] (x|0,1)dx is the cumulative density function of the standard normal distribution, and Ŷ is a sample from the normal distribution with mean vector μₖ and covariance matrix Σₖ. Values of the mean and covariances may be learned from the user responses.

In one or more example fitting agents/methods, the scaling matrix may be a transformed scaling matrix, e.g. by applying a log transform to the elements of the scaling matrix.

The user preference functions may be denoted ƒ or ƒₖ(x; θₖ, Λₖ), where each user preference function ƒₖ(x; θₖ, Λₖ) is associated with environment or state k of K environments/user preference functions. The user preference functions ƒₖ(x; θₖ, Λₖ) may be given by:

$${f}_{k} ( x ; {θ}_{k} , {Λ}_{k} ) = - {( {( x - {θ}_{k} )}^{T} {Λ}_{k} ( x - {θ}_{k} ) )}^{v} , \qquad ( 1 )$$

where x is an M-dimensional vector optionally in the hypercube [0,1]^{M} and that represents the (M) hearing device parameters of the device in the k′th environment, θₖ is the maximizing argument of ƒₖ, Λₖ is a positive definite M×M scaling matrix characterizing user sensitivity to hearing device parameter changes, M is an integer, and v is a real-valued exponent. The real-valued exponent v may be in the range from 0.01 to 0.99. The real-valued exponent v may be less than 0.5 such as in the range from 0.01 to 0.45. The real-valued exponent v may be larger than 0.5 such as in the range from 0.55 to 0.99. In one or more exemplary fitting agents/methods, the real-valued exponent v may be in the range from 0.25 to 0.75, such as 0.5.

The user preference functions ƒₖ(x; θₖ, Λₖ) may be given by:

$$f ( x ; {θ}_{k} , {Λ}_{k} ) = exp ( - {( x - {θ}_{k} )}^{T} {Λ}_{k} ( x - {θ}_{k} ) ) .$$

The variables θₖ, Λₖ are also denoted user preference function parameters, where θₖ is an optimum of the user preference function that corresponds to the optimal user-specific HA parameters and Λₖ∈ [symbol] is a diagonal matrix indicative of a user sensitivity, i.e. ability to distinguish change in the parameter. It is noted that other unimodal user preference functions are also possible.

The fitting agent/one or more processors of the fitting agent is/are configured to update the user model, such as one or more user preference functions ƒₖ(x; θₖ, Λₖ) and/or one or more user response distributions Pᵤ, based on hearing device parameters of the preferred test setting and the environment data. In other words, a user response distribution Pᵤ, may model a user response in one or more environments. A user response distribution may be a weighted user response distribution for all or a subset of the K environments, e.g. where the weights are based on environment probabilities ENVP_k, k=1, . . . , K and distributions of parameters of the user preference functions ƒₖ(x; θₖ, Λₖ).

The fitting agent is configured to update the user model based on hearing device parameters of the preferred test setting, a non-preferred test setting and optionally the environment data. In particular, the fitting agent may be configured to update the user model based on (r, x_ref, x_alt, s). To update the user model may comprise to update the user preference function, or at least parameters thereof and/or to update parameter distributions associated with user preference functions, based on environment data and one or more of the primary test setting, the secondary test setting and the user input of a preferred test setting. In other words, the parameters of the user preference function and/or associated parameter distributions may be updated based on the result of the trial including the primary test setting, the secondary test setting, the preferred test setting of the primary test setting and the secondary test setting, and environment data. To update the user model may comprise to update the user response distribution(s), or at least parameters thereof, based on environment data and one or more of the primary test setting, the secondary test setting and the user input of a preferred test setting. In other words, the parameters of the user preference function may be updated based on the result of the trial including the primary test setting, the secondary test setting, the preferred test setting of the primary test setting and the secondary test setting, and environment data. To update the user model may comprise to update the user response distribution(s), or at least parameters thereof, based on one or more environment probabilities optionally including a first environment probability and a second environment probability. Thus, to update the user model may comprise to update the parameter distribution(s) of user preference function parameters θₖ and/or Λₖ, based on environment data and one or more of the primary test setting, the secondary test setting and the user input of a preferred test setting. In other words, the parameters distributions associated with the user preference functions may be updated based on the result of the trial including the primary test setting, the secondary test setting, the preferred test setting of the primary test setting and the secondary test setting, and environment data. To update the user model, such as to update the parameter distribution(s) of user preference function parameters θₖ and/or Λₖ, may comprise to update the parameter distribution(s) of user preference function parameters θₖ and/or Λₖ, based on one or more environment probabilities optionally including a first environment probability and a second environment probability.

In one or more exemplary fitting agents and/or methods, to update the user model may be based on Bayesian inference. Updating the user model may comprise updating one or more of the parameters of the user preference function and/or user response distribution(s) and/or parameter distributions associated with the user preference functions. Updating the user model may comprise to determine one or more posteriors of parameters of the user preference function(s).

To update the user model may comprise to determine a posterior of the parameters of one or more of, such as a subset of or all the user preference functions, e.g. based on the environment data, a previous parameter posterior, such as the last or current parameter posterior, preferred test setting, and a non-preferred test setting.

Thus, the user preference function for the k′th environment or state may be given as:

$${f}_{k} ( x ; {θ}_{k} , {Λ}_{k} ) = - \sqrt{{( x - {θ}_{k} )}^{T} {Λ}_{k} ( x - {θ}_{k} )} . \qquad ( 4 )$$

The tuning parameters correspond to the location of the optimum of the function, θₖ, which is the optimal hearing device parameters for a user; and to the spread around this optimum, Λₖ, that characterizes user sensitivity to hearing device parameter changes, respectively. The tuning parameters are user-specific and need to be learned in order to provide an optimum or improved user experience. The form of this user preference function has an attractive property that allows for quick narrowing of the search space.

The user model, such as one or more user preference functions, is optionally based on a first assumption and/or a second assumption. The first assumption may be that the user preference functions are unimodal preference functions representative of preference of a user. The second assumption may be that the user can be uncertain about preference, which may be expressed in terms of an additive random variable. A value function uₖ(·) may be defined in order to model user preference uncertainty. The value function uₖ(·) may be defined as

$$( x ) = {f}_{k} ( x ; {θ}_{k} , {Λ}_{k} ) + {ε}_{k} , \qquad ( 5 )$$

where the user uncertainty error εₖ is optionally assumed to have a Gaussian distribution, such as the standard Gaussian distribution, e.g.

$${ε}_{k} ∼ ( 0 , 1 ) \qquad ( 6 )$$

The variance of the Gaussian distribution may be a real value, e.g. in the range from 0 to 1 or larger than 1.

A trial for a user defines a pair of test settings including a primary test setting and a secondary test setting. A user response is obtained to a user's pairwise comparison of test settings from a trial, defined by a pair {x_ref, x_alt} of test settings, where the primary test setting x_ref and the secondary test setting x_alt are the so-called reference and alternative parameter proposal, respectively. In other words, a trial T_n also denoted Dₙ is performed, where each trial T_n comprises or is defined by primary test setting xₙ^{ref}, secondary test setting xₙ^{alt} and preferred/selected test setting rₙ. The index n−1 refers to the previous trial T_n−1.

In the user system, the user response to a trial depends on the user preference. Such connection can be modelled via the model of comparative judgement as follows: P(r|x^{ref}, x^{alt}, ƒ, z=k)=

$${Φ ( f ( {x}^{alt} ; {θ}_{k} , {Λ}_{k} ) - f ( {x}^{ref} ; {θ}_{k} , {Λ}_{k} ) )}^{r} \cdot {Φ ( f ( {x}^{ref} ; {θ}_{k} , {Λ}_{k} ) - f ( {x}^{alt} ; {θ}_{k} , {Λ}_{k} ) )}^{1 - r} \qquad ( 7 )$$

where Φ(·) is the cdf of the standard Gaussian distribution, x^{ref} and x^{alt} are two hearing device parameter sets that the user compares, and z∈{1,2, . . . , K} is the environment/state of the user environment/environment identifier. It is assumed that a user has some uncertainty about his/her choice that is modelled by the additive random noise with the standard Gaussian distribution.

In one or more example fitting agents, the environment data/observed context s is modelled using Gaussian mixture models (GMM), given that there are K possible environments/states of environments:

$$p ( s ) = {\sum }_{z} p ( z ) p ( s \vert z ) = {\sum }_{k = 1}^{K} {π}_{k} \cdot ( s \vert {μ}_{s , k} , {\sum }_{s , k} ) , \qquad ( 8 )$$

where πₖ are mixing probabilities of the environments and μ_{s,k} and Σ_{s,k} are mean values and covariance matrix of the Gaussian distribution, corresponding to the k′th environment. Environment probabilities ENVP_k for the K environments, k=1, . . . , K, may be based on the following:

$$p ( {z}_{t} = k \vert {s}_{t} ) = \frac{{π}_{k} \cdot ( {s}_{t} \vert {μ}_{s , k} , {\sum }_{s , k} )}{{\sum }_{j = 1}^{K} {π}_{j} \cdot ( {s}_{t} \vert {μ}_{s , j} , {\sum }_{s , j} ) ,} \qquad ( 8 A )$$

where sₜ is observed signal/context at time step t and zₜ is environment at this time step.

The complete hearing device system is optionally defined as a sequence of distributions {Pₜ^{a}, Pₜ^{u}}:

$${P}_{t}^{a} {=}^{Δ} {p}_{t}^{a} = {\sum }_{{z}_{t} ∈ \{ 1 , 2 , ... , K \}} {δ ( {x}_{k , t}^{ref} - {x}_{k , t - 1}^{alt} )}^{{r}_{t - 1}} \cdot {δ ( {x}_{k , t}^{ref} - {x}_{k , t - 1}^{ref} )}^{1 - {r}_{t - 1}} \cdot \int p ( {x}_{t}^{alt} , {Λ}_{t} , {z}_{t} \vert {r}_{1}^{t - 1} , {\{ θ , Λ \}}_{1}^{t - 1} , {\{ {x}^{ref} , {x}^{alt} \}}_{1}^{t - 1} , {s}_{1}^{t} ) d {Λ}_{k , t} \qquad ( 9 )$$

$${P}_{t}^{u} {=}^{Δ} {Q}_{t}^{w} = {\sum }_{{z}_{t} ∈ \{ 1 , 2 , ... , K \}} \int \int p ( {θ}_{t} , {Λ}_{t} , {z}_{t} \vert {r}_{1}^{t - 1} , {\{ θ , Λ \}}_{1}^{t - 1} , {\{ {x}^{ref} , {x}^{alt} \}}_{1}^{t - 1} , {s}_{1}^{t} ) \cdot P ( {r}_{t} \vert {θ}_{1}^{t} , {Λ}_{1}^{t} , {z}_{1}^{t} , {s}_{1}^{t} , {r}_{1}^{t - 1} , {\{ {x}^{ref} , {x}^{alt} \}}_{1}^{t} ) d {θ}_{t} d {Λ}_{t}$$

The generative distributions of the fitting agent defined by a sequence of Pₜ^{a} describes the generative process for the next trial {xₜ^{ref}, xₜ^{alt}} as a weighted combination of proposals for possible environments. The generative predictive distribution Pₜ^{u} characterizes the user response distribution for some next trial {xₜ^{ref}, xₜ^{alt}}. The primary test settings/reference proposals x_{k,t}^{ref} are defined in a deterministic way based on the user response in the previous trial. Secondary test settings/alternative proposals x_{k,t}^{alt} are generated from the user preference posterior, since from the optimization point of view the goal of the fitting agent is get to the optimum as quickly as possible and, thus, provide a better option than a reference proposal, with high probability. Clearly, this option would correspond to the user preference or the fitting agent estimate of it.

Finally, the fitting agent strategy for the sequential trial design in the hearing device system, defined by these distributions {Pₜ^{a}, Pₜ^{a}}, may be to generate a sequence of trials t=1,2, . . . , T, based on the history of interactions with the user in some environments according to the following rule, where I(·) denotes mutual information:

$$\{ {x}_{t}^{ref} , {x}_{t}^{alt} \} = arg {max}_{\{ {x}^{ref} , {x}^{alt} \} : {P}_{t}^{a}} {\sum }_{{z}_{t} ∈ \{ 1 , 2 , ... , K \}} \text{} p ( \text{} {z}_{t} \vert {s}_{t} ) \text{} I ( \text{} R ; \\ {θ}_{t} \vert {z}_{1}^{t} , {s}_{1}^{t} , {R}_{1}^{t - 1} , {\{ θ , Λ \}}_{1}^{t - 1} , {\{ {x}^{ref} , {x}^{alt} \}}_{1}^{t - 1} , \{ {x}^{ref} , {x}^{alt} \} ) \qquad ( 10 )$$

In order to generate required trials, the fitting agent has to assess the user model first. Hence, the fitting agent has to sequentially assign predicted distribution Qₜ, associated with the user model such that the difference between Qₜ, and the true distribution of the user response is minimized for all system interactions, and this difference is given by the normalized weighted KL-divergence after T interactions:

$$D ( Q , \{ {x}^{ref} , {x}^{alt} \} , T ) {=}^{Δ} \frac{1}{T} \int \int w ( θ , Λ ) 𝔻 ( {P}_{T}^{u} ‖ {Q}_{T} ) d θ d Λ \qquad ( 11 )$$

with some weighting function w(θ, Λ). For the present hearing device system, defined by {Pₜ^{a}, Pₜ^{a}}≙{pₜ^{a}, Qₜ^{w}} as above, this normalized KL-divergence may be given by the following expression:

$$D ( {Q}^{w} , \{ {x}^{ref} , {x}^{alt} \} , T ) = \frac{1}{T} \text{} {\sum }_{t = 1}^{T} \text{} {\sum }_{{z}_{t} ∈ \{ 1 , 2 , ... , K \}} \text{} p ( \text{} {z}_{t} \vert {s}_{t} ) \text{} I ( \text{} R ; \\ {θ}_{t} \vert {z}_{1}^{t} , {s}_{1}^{t} , {R}_{1}^{t - 1} , {\{ θ , Λ \}}_{1}^{t - 1} , {\{ {x}^{ref} , {x}^{alt} \}}_{1}^{t} ) , \qquad ( 12 )$$

where Qₜ^{w}, t=1,2, . . . , T are distributions minimizing the normalized weighted KL-divergence, and the weighting distribution corresponds to the posterior distribution of the user preference function parameters.

It is seen that the metric in (12) can be written as a sum of mutual information for each interaction step and may characterize the fitting agent uncertainty about the user system. Therefore, the fitting agent goal of generating informative trials (test setting) is translated into finding a trial/test setting that can maximally reduce the remaining system uncertainty. This metric also allows efficient monitoring of the system performance.

Finally, after the fitting agent strategy has been defined, the posterior of the user preference(s), p(θₜ, Λₜ, zₜ|r₁^{t-1},{θ, Λ}₁^{t-1},{x^{ref} x^{alt}}₁^{t-1}, s₁^{t}) must be learned. This step is also a part of the fitting agent strategy, as the posterior of the user preference is also the weighting distribution used in the generative predictive user response distribution. In one or more example fitting agents, GMM is used to estimate the environment state and then Bayesian variational inference is optionally used to further infer the user preference for hearing device parameters for the K environments.

In one or more example fitting agents, to obtain environment data comprises to obtain audio data and optionally determining the environment data based on the audio data and/or including the audio data in the environment data. In other words, one or more processors of the fitting agent may be configured to obtain audio data and determining the environment data or at least one or more environment parameters based on the audio data. Audio data may comprise first audio data representing or being indicative of audio recorded by one or more microphones of a hearing device of the user. Audio data may comprise second audio data representing or being indicative of audio recorded by one or more microphones of an accessory device or accessory devices of the user. Audio data may comprise third audio data representing or being indicative of audio wirelessly transmitted to a hearing device of the user. For example, the fitting agent may be configured to classify the environment based on hearing device audio and set one or more environment identifiers and/or environment probabilities of the environment data accordingly.

In one or more example fitting agents, to obtain environment data comprises to receive user input indicative of the environment, e.g. via a user interface of an accessory device. Thus, the user may select and indicate the present environment via accessory device, e.g. from a list of environments presented on a touch-display of the accessory device.

In one or more example fitting agents, the one or more processors are configured to obtain a first environment probability of a first environment and/or a second environment probability of a second environment based on the environment data, and wherein to obtain a test setting and/or to update the user model is optionally based on the first environment probability and/or the second environment probability. In other words, one or more processors of the fitting agent may be configured to obtain, such as one or more of determine, estimate, receive, and retrieve, a first environment probability also denoted ENVP_1 of a first environment ENV_1 and/or a second environment probability ENVP_2 of a second environment ENV_2, and optionally obtain a test setting and/or update the user model, such as the first user preference function and/or the second user preference function, based on the first environment probability and/or the second environment probability. The environment data optionally comprises an environment probability ENVP_k of or associated with each environment ENV_k of the K environments, where an environment probability ENVP_k is a probability of the present environment being the corresponding environment ENV_k. For example, ENVP_2=0.5 may indicate a probability of 0.5 that the present environment is the second environment ENV_2. For example, ENVP_2=1.0 may indicate that the present environment is the second environment ENV_2.

In other words, the fitting agent, such as one or more processors of the fitting agent may be configured to obtain, such as one or more of determine, estimate, receive, and retrieve one or more environment probabilities, such as K environment probabilities, indicative of probability that the present environment is a corresponding environment, e.g. based on the environment data, such as one or more environment parameters.

In one or more exemplary fitting agents, the environment probabilities are given by equation 8A.

The first environment probability may be indicative of a probability that the present environment is a first environment. The first environment may be an environment of a first type, such as an environment characterized by a low SNR and high sound level/power, such as a cocktail party or a concert.

The second environment probability may be indicative of a probability that the present environment is a second environment. The second environment may be an environment of a second type, such as an environment characterized by low SNR and medium sound level/power.

In one or more example fitting agents, the plurality of user preference functions comprises K user preference functions, wherein K is 2, 3, or larger than 3. The number K of user preference functions may be in the range from 5 to 25, such as from 5 to 10, e.g. 7. In one or more example fitting agents, to update the user model comprises to select a user preference function to be updated from the K user preference functions based on the environment data and update a posterior of the parameters of the user preference function to be updated based on the test setting and the user input of preferred test setting.

In one or more example fitting agents, to update the user model comprises to select a plurality of user preference functions, such as 2, 3, 4, or more to be updated from the K user preference functions based on the environment data and update posteriors or posterior distributions of the parameters of the plurality of user preference functions to be updated based on the test setting and the user input of preferred test setting. In one or more example fitting agents, to update the user model comprises to update the K user preference functions and parameter distributions associated therewith, or to update the user preference functions and parameter distributions associated therewith having an environment probability larger than 0.

In one or more example fitting agents, to obtain environment data comprises to determine an environment identifier and/or to obtain one or more environment probabilities, such as K environment probabilities, e.g. using a Gaussian mixture model. To update the user model may be based on the environment identifier and/or one or more environment probabilities, such as environment probabilities for environments ENV_k, k=1, 2, . . . , K. For example, to update the user model based on the environment identifier may comprise to select a user preference function and associated parameter distribution for update based on or in accordance with the environment identifier and update the user preference function and associated parameter distribution selected for update based on the preferred test setting and optionally the non-preferred test setting.

In one or more example fitting agents, the fitting agent is configured to determine whether a test criterion based on the environment data is satisfied, wherein the fitting agent is configured to, in accordance with a determination that the test criterion is satisfied, perform: obtain a test setting comprising a primary test setting and a secondary test setting for the hearing device; present the test setting to the hearing device user; obtain a user input of a preferred test setting indicative of a preference for either the primary test setting or the secondary test setting; and update the user model based on hearing device parameters of the preferred test setting and the environment data.

Accordingly, a test criterion may be used to determine, when a user's input is needed to improve the accuracy of the user preference functions. Hereby is provided for efficient and to-the-point training of the user preference functions, e.g. such that a user is only prompted for input when needed, thereby reducing and/or optimizing the number of user interactions.

In one or more example fitting agents, the test criterion is based on an uncertainty metric indicative of user preference uncertainty in the present environment. For example, the test criterion may be satisfied or at least partly satisfied if it is determined that the user preference function associated with the present environment is not precise enough, e.g. has a high degree of uncertainty, for example being reflected in an uncertainty metric being larger than a threshold.

In one or more example fitting agents, to obtain a test setting comprising a primary test setting and a secondary test setting for the hearing device comprises to determine the secondary test setting based on the environment data, e.g. as described above in relation to equations (10)-(12).

In one or more example fitting agents, the fitting agent is configured to determine and update hearing device parameters of the hearing device based on the updated user model and/or the environment data, such as environment probabilities of the environment data.

The environment model may be a Gaussian mixture model, e.g. with a number K of predefined maximum clusters/states/environments. K is set upfront, but some clusters might be empty. In other words, a cluster of the environment may be representative of an environment.

The environment model may be a non-parametric Bayesian mixture model, such as a Dirichlet process Gaussian mixture model also denoted DP-GMM. A non-parametric Bayesian mixture model models an unknown infinite number of environmental clusters.

In one or more examples, to update the environment model may comprise increasing the number of clusters, such as increase the number of populated or non-empty clusters of the environment model.

In one or more examples, to update the environment model may comprise decreasing the number of clusters, such as decrease the number of populated or non-empty clusters of the environment model. In other words, to update the environment model may comprise increasing the number of empty clusters.

The fitting agent, such as one or more processors of the fitting agent, is configured to obtain environment data indicative of a present environment. The environment data may comprise signal vector or signal matrix denoted s of one or more input signals and/or other contextual information or any parameter determined based thereon, such as signal-to-noise ratio (SNR) and power.

An environment, such as an acoustic environment, may be characterized by one or more of a first environment parameter, such as input signal level or input power, and a second environment parameter, such as SNR, for one or more input signals. In other words, the environment data may comprise a (first) SNR and/or a (first) power of a (first) input signal. Thus, to obtain environment data indicative of a present environment may comprise to obtain, such as determine or receive, one or more environment parameters optionally including a (first) SNR and/or a (first) power of a (first) input signal.

The fitting agent, such as one or more processors of the fitting agent, is configured to determine a first initial environment probability of a first environment and/or a second initial environment probability of a second environment, e.g. based on the environment data and the environment model.

The fitting agent, such as one or more processors of the fitting agent, is configured to obtain, such as one or more of determine, receive, and retrieve, a test setting comprising a primary test setting and a secondary test setting for the hearing device, e.g. based on the first initial environment probability and/or the second initial environment probability.

The primary test setting is also denoted reference proposal and the secondary test setting is also denoted alternative proposal. The test setting is also referred to as a trial. Each trial, denoted by {θref,θalt}, comprises the reference proposal θref and the alternative proposal θalt.

The fitting agent is configured to obtain a primary test setting also denoted θref, x_ref or x^{ref} for the hearing device. The primary test setting is a vector comprising M hearing device parameters for the hearing device. The hearing device parameters may comprise one or more of filter coefficients, compressor settings, gains, or other parameters relevant for the operation of or signal processing in the hearing device. The primary test setting may be based on and/or dependent on the present environment, such as based on the first initial environment probability and/or the second initial environment probability. In other words, the primary test setting may be based on and/or dependent on the environment data.

The primary test setting may be based on and/or dependent on one or more environment probabilities including a first environment probability ENVP_1 indicative of the present environment being a first environment and/or a second environment probability ENVP_2 indicative of the present environment being a second environment. The primary test setting may be a weighted combination (with environment probabilities as weights ENVP_k) of test settings for the environments ENV_k, k=1, 2, . . . , K.

The fitting agent is configured to obtain a secondary test setting also denoted θalt, x_alt or x^{alt} for the hearing device. The secondary test setting x_ref is a vector comprising M hearing device parameters for the hearing device. The hearing device parameters may comprise one or more of filter coefficients, compressor settings, gains, or other parameters relevant for the operation of or signal processing in the hearing device. The secondary test setting may be based on and/or dependent on the present environment, such as based on the first initial environment probability and/or the second initial environment probability. In other words, the secondary test setting may be based on and/or dependent on the environment data.

The secondary test setting may be based on and/or dependent on one or more environment probabilities including a first environment probability ENVP_1 indicative of the present environment being a first environment and/or a second environment probability ENVP_2 indicative of the present environment being a second environment. The secondary test setting may be a weighted combination (with environment probabilities as weights ENVP_k) of test settings for the environments ENV_k, k=1, 2, . . . , K.

The fitting agent, such as one or more processors of the fitting agent, is configured to present the test setting to the hearing device user. To present the test setting to the hearing device user comprises presenting the primary test setting and/or the secondary test setting to a user. To present the primary test setting and the secondary test setting to a user optionally comprises to output a primary test signal according to the primary test setting. The primary test signal may be an audio signal. The primary test signal may be output via loudspeaker or receiver of a hearing device. To present the primary test setting and the secondary test setting to a user optionally comprises to generate the primary test signal according to the primary test setting in accessory device and to stream the primary test signal from accessory device to hearing device. To present the primary test setting and the secondary test setting to a user optionally comprises to transmit a control signal indicative of primary test signal/primary test setting from accessory device to hearing device. The control signal may include primary test setting. To present the primary test setting and the secondary test setting to a user may comprise to generate the primary test signal according to the control signal in the hearing device, e.g. based on primary test setting of the control signal.

The fitting agent, such as one or more processors of the fitting agent, is configured to obtain, such as receive and/or detect, a user input of a preferred test setting indicative of a preference for either the primary test setting or the secondary test setting. In other words, the user gives a binary response r to indicate his/her preferred set of HA parameters out of the two proposals θref and θalt.

The non-preferred test setting is the primary test setting or the secondary test setting not being selected as a preferred test setting. Accordingly, a hearing device and/or accessory device(s) implementing the fitting agent or at least a part of the fitting agent may comprise one or more user interfaces for obtaining, such as receiving and/or detecting, a user input. For example, the hearing device may comprise a user interface receiving a user input. The user interface of the hearing device may comprise one or more buttons, an accelerometer and/or a voice control unit. The accessory device may comprise a user interface. The user interface of the accessory device may comprise a touch sensitive surface, e.g. a touch display, and/or one or more buttons. The user interface of the accessory device may comprise a voice control unit. The user interface of the hearing device may comprise one or more physical sliders, knobs and/or push buttons. The user interface of the accessory device may comprise one or more physical or virtual (on-screen) sliders, knobs and/or push buttons.

The fitting agent is configured to obtain, such as detect, a user input of a preferred/selected test setting indicative of a preference for either the primary test setting or the secondary test setting. In the fitting agent, to detect a user input of a preferred test setting indicative of a preference for either the primary test setting or the secondary test setting may comprise prompting the user for the user input, e.g. by a beep tone signal or voice signal from the hearing device and/or a visual, haptic and/or audio prompt from an accessory device. Detecting a user input may be performed on the hearing device, e.g. by a user activating a button and/or an accelerometer (e.g. single or double tapping the hearing device housing) in the hearing device. To detect a user input may be performed on an accessory device, e.g. by a user selecting a user interface element representative of the preferred test setting, e.g. on a touch-sensitive display of the user accessory device.

The fitting agent, such as one or more processors of the fitting agent, is configured to update the user model for provision of an updated user model e.g. based on the preferred test setting and the environment data.

The fitting agent, such as one or more processors of the fitting agent, may be configured to determine hearing device parameters based on the updated user model and output the hearing device parameters, such as transmit the hearing device parameters to the hearing device.

The fitting agent may be implemented in one or more electronic devices, such as a hearing device and/or accessory device(s). The fitting agent is an electronic fitting agent. The fitting agent or at least parts thereof may be implemented in an accessory device or a plurality of accessory devices, such as in a smartphone optionally in combination with a smartwatch. The fitting agent may be for/part of one or more of optimizing, determining, fitting, tuning, and modelling, such as determining hearing device parameters of a hearing device. Implementing the fitting agent in accessory device(s), such as a smartphone optionally in combination with a smartwatch, may be advantageous in providing a more smooth user input and user experience. Further, implementing part(s) of the fitting agent in accessory device(s) may be advantageous in providing a more power efficient method from the perspective of the hearing device. The fitting agent or at least parts thereof may be implemented in a server device and/or in a fitting device.

The present disclosure relates to a fitting agent for a hearing device, and in particular to a fitting agent for one or more of optimizing, determining, fitting, and tuning hearing device parameters of a hearing device.

In one or more examples, a fitting agent for a hearing device system comprising a hearing device worn by a hearing device user is disclosed, wherein the fitting agent comprises one or more processors configured to initialize a user model and an environment model, the user model comprising one or a plurality of user preference functions and associated user response distribution; obtain environment data indicative of a present environment; determine a first initial environment probability of a first environment and a second initial environment probability of a second environment based on the environment data and the environment model, obtain a test setting comprising a primary test setting and a secondary test setting for the hearing device based on the first initial environment probability and the second initial environment probability; present the test setting to the hearing device user; obtain a user input of a preferred test setting indicative of a preference for either the primary test setting or the secondary test setting; and update the user model for provision of an updated user model based on the preferred test setting and the environment data. The updated user model may be based on the trial/test setting.

In one or more examples, the one or more processors are configured to estimate a personalized environment probability of the present environment based on the updated user model.

In one or more examples, the personalized environment probability is a probability of one or more user preference clusters in the domain of hearing device parameters. In other words, the personalized environment probability may comprise one or more probabilities, where each probability is a posterior probability of a user preference cluster. We also refer to these probabilities as reliability or soft information.

In one or more examples, the one or more processors are configured to update the environment model for provision of an updated environment model, e.g. based on the personalized environment probability and the environment data. The update of the environment model for provision of an updated environment model may be performed before, in parallel to and/or after updating the user model. To update the environment model may comprise outputting, such as transmitting and/or storing the updated environment model. The updated model environment model may be transmitted to a server device and stored therein, e.g. for later use and update as the environment model. The updated environment model may be stored in the accessory device, e.g. for later use and update as the environment model, e.g. in a memory of the accessory device.

In one or more examples, to update the environment model comprises to update an environment distribution function of the environment model based on the personalized environment probability and the environment data. In other words, the environment model may comprise an environment distribution function.

In one or more examples, the environment model is a Gaussian Mixture Model also denoted GMM. The environment model may be a non-parametric Bayesian mixture model, such as a Dirichlet process Gaussian mixture model also denoted DP-GMM.

For example, given the environment data represented by signal vector sₜ and the GMM model of the environment, the reliability information about the present state of the environment can be obtained as:

$$p ( {\tilde{z}}_{t} = k \vert {s}_{t} ) = \frac{{π}_{t , k} \cdot ( {s}_{t} \vert {μ}_{s , k} , {\sum }_{s , k} )}{{\sum }_{j = 1}^{K} {π}_{t , j} \cdot ( {s}_{t} \vert {μ}_{s , j} , {\sum }_{s , j} )} .$$

Where \tilde{z}ₜ denotes the state of the environment, μ_{s,k} and Σ_{s,k} denote the parameters (mean and covariance) of the signal distribution of k-th cluster, and π_{t,k} the enrivomental mixing probabilities.

We also use cₜ,mₖ, Wₖ to denote the state of the enviroments as well as paramters of the signal distribution, respectively, in the text. Here Wₖ is precistion matrix, which corresponds to the inverse of covariance matrix. The use of notation will be clear from the context. Also index 0 for parameters indicates hyperparameters.

Depending on the context, while referring to the reliability or soft information, we refer to prior or the posterior probability of the cluster in the signal/environmental domain or user preference/parameter domain. Prior probability in the user preference domain is the posterior probability from the signal/environmental domain; posterior information in the user preference/parameter domain is a personalized environment probability.

This reliability/reliability information is used for mapping from environment space to the parameter space, where the user preferences depend on the environment. In the parameter space, this reliability information is regarded as prior information about optimal preference cluster. In other words, this reliability information is a mixture coefficient of the GMM, corresponding to the parameter space.

After the user-agent interaction during the trial in this environment, i.e. present test setting and obtain user input of preferred test setting, the fitting agent updates the user model parameter posterior given kth environment, e.g., using assumed density filtering (ADF). Since the approximation family is Gaussian, the full parameter posterior is again a GMM.

Therefore, based on the approximated parameter posterior qₜ(ϕₜ|zₜ), posterior for the preference clusters for the preferred parameter settings, obtained after the agent-user interaction, i.e., xₜ^{ref}, may be obtained as

$$p ( {\tilde{z}}_{t} = k \vert {x}_{t}^{ref} ) = \frac{p ( {\tilde{z}}_{t} = k \vert {s}_{t} ) \cdot {q}_{t} ( {x}_{t}^{ref} \vert {z}_{t} = k )}{{\sum }_{j = 1}^{K} p ( {\tilde{z}}_{t} = j \vert {s}_{t} ) \cdot {q}_{t} ( {x}_{t}^{ref} \vert {z}_{t} = k )} = \frac{p ( {\tilde{z}}_{t} = k \vert {s}_{t} ) \cdot ( {x}_{t}^{ref} \vert {μ}_{ϕ , t , k} , {\sum }_{ϕ , t , k} )}{{\sum }_{j = 1}^{K} p ( {\tilde{z}}_{t} = j \vert {s}_{t} ) \cdot ( {x}_{k , t}^{ref} \vert {μ}_{ϕ , t , j} , {\sum }_{ϕ , t , j} )} .$$

The one or more processors may update the environment model for provision of an updated environment model based on the personalized environment probability and the environment data by using the environment data (observed signal sₜ) and reliability information p(\tilde{z}ₜ=k|xₜ^{ref}) from the parameter space. Thus, a mapping in the opposite direction from the user preference parameter space into environment space is obtained.

In one or more examples, to update the environment model is based on an expectation maximization algorithm, such as online EM. In other words, the environment model for individual components can be updated using online or recursive EM, e.g. as:

$${π}_{t + 1 , k} = {π}_{t , k} + v \cdot ( p ( {\tilde{z}}_{t} = k \vert {x}_{t}^{ref} ) - {π}_{t , k} )$$

$${μ}_{s , t + 1 , k} = {μ}_{s , t , k} + v \cdot \frac{p ( {\tilde{z}}_{t} = k \vert {x}_{t}^{ref} )}{{π}_{t , k}} \cdot ( {s}_{t} - {μ}_{s , t , k} )$$

$${\sum }_{s , t + 1 , k} = {\sum }_{s , t , k} + v \cdot \frac{p ( {\tilde{z}}_{t} = k \vert {x}_{t}^{ref} )}{{π}_{t , k}} \cdot ( ( {s}_{t} - {μ}_{s , t , k} ) \cdot {( {s}_{t} - {μ}_{s , t , k} )}^{T} - {\sum }_{s , t , k} ) ,$$

where 0<v<1 is a forgetting factor.

Other variants of online EM are also possible. For example, one or more EMs with time-dependent forgetting factor and/or regularization may be implemented.

Note that with some modifications, e.g. by introducing Dirichlet priors on the number of clusters, i.e., K, π_{t+1,k} can also be used to remove clusters.

In one or more examples, to obtain environment data comprises obtaining position data, e.g. from a GPS unit of the accessory device, the position data indicative of a user position, and determine the environment data based on the position data.

In one or more examples, to obtain environment data comprises obtaining audio data indicative of sound in the present environment and determine the environment data based on the audio data.

In one or more examples, to obtain environment data comprises obtaining context data indicative of the surroundings and/or activity of the user and determine the environment data based on the context data. Context data may comprise one or more of calendar data, activity data or motion data, and the one or more processors may be configured to determine the environment data based on one or more of calendar data, activity data and motion data. The activity data may be indicative of an activity of the user, e.g. watching TV, in a meeting, at a concert, having dinner, etc. Context data may be indicative of the context in which the user is in, such as indicative of a user's location, position, movement, temperature, pulse, or other data relevant for the environment. For example, the context data may comprise location data, e.g. GPS coordinates, and/or movement data, such as accelerometer data. The context data may comprise calendar data, and the environment data may be based on the calendar data. The context data may comprise sensor data, e.g. from one or more sensors of an accessory device and/or from one or more sensors of the hearing device. The context data may comprise hearing device data transmitted from the hearing device, such as one or more program identifiers, one or more operating parameters, and/or one or operating mode identifiers of the hearing device.

In one or more example fitting agents, to obtain environment data comprises to receive user input indicative of the environment, e.g. via a user interface of an accessory device. Thus, the user may select and indicate the present environment via accessory device, e.g. from a list of environments presented on a touch-display of the accessory device.

In one or more examples, to initialize the environment model comprises to obtain a user profile and initialize the environment model based on the user profile.

In one or more examples, the user profile comprises one or more of age, gender, hearing loss degree, and activity level, and wherein to initialize the environment model is based on one or more of age, gender, hearing loss degree, and activity level.

In one or more examples, to initialize the environment model comprises to obtain an environment profile and initialize the environment model based on the environment profile. To obtain an environment profile may comprise to select, determine, and/or retrieve a default or initial environment. To obtain an environment profile may comprise to determine the environment profile based on audio data from one or more microphones and/or a user input, such as via a questionnaire.

The fitting agent may be configured to one or more of use, implement, store, infer, and employ the updated user model as the user model in the fitting agent.

The fitting agent may be configured to one or more of use, implement, store, infer, and employ the updated environment model as the environment model in the fitting agent.

The present disclosure provides a context/environment-aware preference learning system that can learn a user's preference in different environments. In the following Section A, an example preference learning system for a single environment is disclosed.

In Section B, an example preference learning system for multiple environments is disclosed. Section C describes an example environment model, and Section D provides a model summary.

### A. Preference Learning System

The preference learning system is modeled as two sub-systems, a fitting agent and a user, interacting with each other. The parameters of the fitting agent are denoted its internal state. In one interaction cycle, the fitting agent proposes a trial to the user based on its internal state and the history of interactions. Each trial, denoted by {θref,θalt}, contains a so-called reference and alternative proposal. The user provides a binary response r to indicate his/her preferred set of hearing device parameters out of two proposals. More precisely, the user response to a trial {θref, θalt} may be defined as

$$R ≡ \{ \begin{matrix}0 , & if {θ}^{ref} ≻ {θ}^{alt} \\ 1 , & if {θ}^{alt} ⪰ {θ}^{ref}\end{matrix} ,$$

where θref>θalt means that the user prefers the reference proposal over the alternative proposal; and where R denotes a random variable, corresponding to the user response, while r denotes its realization. By interacting with the user, the fitting agent also denoted agent aims at estimating the optimal preferences of the user, given the user's responses, in as few trials as possible. In order to define a user response model and define efficient interactions, two assumptions about user preferences are made.

First, an optimal user preference exists and is described by a unimodal preference function f(x;θ,Λ), given by

$$f ( x ; θ , Λ ) = - \sqrt{{( x - θ )}^{T} Λ ( x - θ )} ,$$

where x denotes the hearing device tuning parameters, θ∈[0,1]^{D} denotes the optimal tuning parameters corresponding to the user preference, and Λ=diag([λ₁, . . . , λ_{D}]), λ_{d}∈R⁺, d=1, 2, . . . , D denotes a users' sensitivity to parameter changes.

Note, that for a hearing device also denoted HA with D tuning parameters, it is common that different parameters have different scales, therefore, to guarantee that θ∈[0, 1]^{D}, each parameter is normalized to [0, 1]. Second, a user can be uncertain about his/her preference for one of the two proposals, which is represented by an additive random variable ε˜N (0, 1).

Then the user response model is given by a binomial-probit model:

$$P ( r \vert {θ}^{ref} , {θ}^{alt} , θ , Λ ) = Ber ( r \vert Φ ( f ( {θ}^{alt} ; θ , Λ ) - f ( {θ}^{ref} ; θ , Λ ) ) ) ,$$

where Φ(·) is the cumulative distribution function (CDF) of the standard normal distribution.

The true values of parameters θ and Λ of the user preference function (2) are unknown. In order to learn those parameters, the agent proposes trials and based on the obtained information from the user infers θ and Λ.

Observe that tuning and sensitivity parameters θ,Λ are bounded. For modeling convenience, this constraint is relaxed by introducing the following transformation:

$$ϕ = {[ {Φ}^{- 1} ( θ ) , ln Λ ]}^{T} ,$$

where Φ⁻¹(·) is the element-wise inverse of standard normal CDF and In Λ≙[In λ₁, . . . , In λ_{D}]. This transformation ensures that, during the learning procedure, the user preference remains in the hypercube [0, 1]^{D} and the user sensitivity in the positive real half-space.

The agent has two components. One component corresponds to the generative distributions Pₜ^{a} for a trail under the internal state φ, given previous observations and trials. The other component is the predictive user response distribution Pₜ^{u} used by the agent to model the user response. In summary, a preference learning system model is defined by a combination of two sub-systems {Pₜ^{a}, Pₜ^{u}}, an agent and a user, given by a sequence of distributions:

$$\begin{matrix}{P}_{t}^{a} = p ( \{ {θ}_{t}^{ref} , {θ}_{t}^{alt} \} \vert ϕ , {r}_{1 : t - 1} , {\{ {θ}^{ref} , {θ}^{alt} \}}_{1 : t - 1} ) \\ {P}_{t}^{u} = P ( {r}_{t} \vert ϕ , {r}_{1 : t - 1} , {\{ {θ}^{ref} , {θ}^{alt} \}}_{1 : t - 1} , \{ {θ}_{t}^{ref} , {θ}_{t}^{alt} \} ) ,\end{matrix}$$

For t=1, 2, . . . where Pₜ^{a} is the generative proposal distribution under internal state φ, given previous sequence of responses r_{1:t-1}=(r₁, . . . , rₜ₋₁) and trials {θref, θalt}_{1:t-1}; and Pₜ^{u} is predictive PMF of the user response to a new trial {θref,θalt}, under this internal state, given r and {θref, θalt}_{1:t-1}.

### B. State-Dependent Preference Learning System

In order to extend the preference learning system to be capable of learning multiple user preferences, we assume that user preferences depend on the acoustic environments. We use the environmental state to denote different acoustic environments. Using the environmental state we define a state-dependent user preference function as

$${f}_{k} ( x ; θ , Λ ) = - \sqrt{{( x - {θ}_{k} )}^{T} {Λ}_{k} ( x - {θ}_{k} )} ,$$

where θₖ and Λₖ are parameters, corresponding to the state k and θ, Λ is the collection of all parameters corresponding to all states. Therefore, at time t, the state-dependent user response model becomes

$$P ( {r}_{t} \vert {θ}_{t}^{ref} , {θ}_{t}^{alt} , θ , Λ , {c}_{t} = k ) = Ber ( {r}_{t} \vert Φ ( {f}_{k} ( {θ}_{t}^{alt} ; θ , Λ ) - {f}_{k} ( {θ}_{t}^{ref} ; θ , Λ ) ) ) ,$$

where cₜ∈N is the state of the environment at time t and θref,θalt are the reference and alternative proposals for this state. Using these two formulas, the state-dependent preference learning system can be represented by

$$\begin{matrix}{P}_{t}^{a} = p ( \{ {θ}_{t}^{ref} , {θ}_{t}^{alt} \} \vert ϕ , {c}_{t} = k , {r}_{1 : t - 1} , {\{ {θ}^{ref} , {θ}^{alt} \}}_{1 : t - 1} ) \\ = p ( \{ {θ}_{t}^{ref} , {θ}_{t}^{alt} \} \vert {ϕ}_{k} , {r}_{1 : t - 1} , {\{ {θ}^{ref} , {θ}^{alt} \}}_{1 : t - 1} )\end{matrix}$$

$$\begin{matrix}{P}_{t}^{u} = P ( {r}_{t} \vert ϕ , {c}_{t} = k , {r}_{1 : t - 1} , {\{ {θ}^{ref} , {θ}^{alt} \}}_{1 : t - 1} , {\{ {θ}^{ref} , {θ}^{alt} \}}_{t} ) \\ = P ( {r}_{t} \vert ϕ , {r}_{1 : t - 1} , {\{ {θ}^{ref} , {θ}^{alt} \}}_{1 : t - 1} , {\{ {θ}^{ref} , {θ}^{alt} \}}_{t} ) ,\end{matrix}$$

where φ is the collection of φk for all states k=1,2, . . . , and φk=[Φ⁻¹(θk),lnΛk]. To complete the model, the normal distribution for the prior of φk is assumed, i.e.,

$$p ( {ϕ}_{k} ) = 𝒩 ( {ϕ}_{k} \vert {μ}_{ϕ}^{k} , {Σ}_{ϕ}^{k} ) .$$

### C. Environment Model

The HA tuning parameters are used to enhance the acoustic signal for users to provide them with good sound quality. From this point of view, the state of an environment should be inferred from the acoustic signals produced in this environment. Thus, an acoustic signal model is implemented to supply environment data to the preference learning system. The inferred environment state serves as the state cₜ in Section B above.

The HA will record the acoustic signal from the environment, then convert the signal to a signal feature sₜ on time step t. The environment model may be defined in this feature space. Moreover, a correspondence between signal feature space and user preference is assumed, i.e., for each cluster in the feature space, there is a corresponding user preference. Therefore, the environment state cₜ is shared by the acoustic signal model and the agents.

The signal features sₜ are modeled using a Gaussian mixture model (GMM). In this model, each component represents a signal cluster, and each k^{th} component is modeled by a Gaussian distribution with mean vector mₖ and precision matrix Wₖ to represent different signal clusters. Thus, the GMM may be defined as

$$p ( {𝓈}_{t} \vert m , W , {c}_{t} = k ) = 𝒩 ( {𝓈}_{t} \vert {m}_{k} , {W}_{k}^{- 1} ) ,$$

where m=[m₁,m₂, . . . ,m_{∞}] and W=[W1, . . . ,W_{∞}]. The same Normal-Wishart distribution is assumed as a prior for all parameters mₖ and Wₖ of the mixture components k=1,2, . . . ,K:

$$p ( {m}_{k} , {W}_{k} ) = 𝒩𝒲 ( {m}_{k} , {W}_{k}^{- 1} \vert {m}_{0} , {λ}_{0} , {W}_{0} , {v}_{0} ) ,$$

where m₀, λ₀, v₀, W₀ are four parameters of the Normal-Wishart Distribution. Observe that GMM has a predefined number of clusters. However, the number of the relevant clusters for a user is typically unknown and can change over time. To address this problem, we propose to use a DP as the prior on the mixing components. Given the mixing probability vector π, the cluster label cₜ follows a categorical distribution

$$p ( {c}_{t} \vert π ) = Cat ( {c}_{t} \vert π ) .$$

This mixing probability π describes how probable each state of the environment is and follows the GEM distribution

$$p ( π ) = GEM ( π \vert α ) ,$$

where the GEM distribution is a limit distribution of Dirichlet distribution

$$GEM ( α ) = {lim}_{K \to ∞} Dir ( α / K , … ) ,$$

named after Griths, Engen and McCloskey. Its samples contain an infinite number of elements. Here a is the concentration parameter.

### D. Model Summary

The context-aware preference learning system can be defined as outlined in Sections B and C optionally together with the DP prior for environmental states cₜ, and priors for the signal model and the agents. To summarize, the context-aware preference learning system can be defined as the following generative model:

$$\begin{matrix}π \sim GEM ( α ) \\ {c}_{t} \vert π \sim Cat ( π ) \\ {m}_{k} , {W}_{k} \sim 𝒩𝒲 ( {m}_{0} , {λ}_{0} , {W}_{0} , {v}_{0} ) \\ {𝓈}_{t} \vert {c}_{t} = k , m , W \sim 𝒩 ( {m}_{k} , {W}_{k} ) \\ {ϕ}_{k} \sim 𝒩 ( {μ}_{ϕ} , {Σ}_{ϕ} ) \\ \{ {θ}_{t}^{ref} , {θ}_{t}^{alt} \} \vert {c}_{t} = k , ϕ \sim {P}_{t}^{a} \\ {r}_{t} \vert \{ {θ}_{t}^{ref} , {θ}_{t}^{alt} \} , {c}_{t} = k , ϕ \sim Ber ( Φ ( {f}_{k} ( {θ}_{t}^{alt} ; θ , Λ ) - {f}_{k} ( {θ}_{t}^{ref} ; θ , Λ ) ) ) .\end{matrix}$$

It is noted that descriptions and features of fitting agent functionality also applies to methods and vice versa.

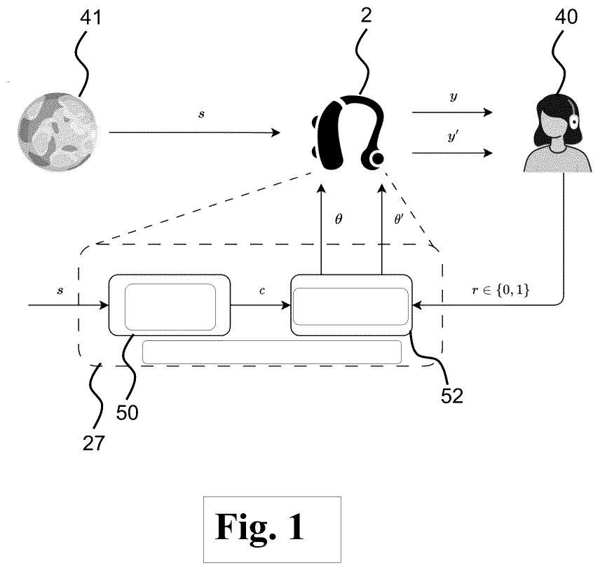

FIG. 1 shows a schematic overview of the fitting agent with a context-aware preference learning system. A signal s is received by the hearing device 2 and the fitting agent 27, e.g. implemented in accessory device, the fitting agent passing environment data to the environment model 50. The environment model 50 infers the current environment state c for the preference learning system 52. Based on the environment 41/state and previous trials and user responses, the preference learning system 52 including user model proposes two tuning parameter sets θ (first primary test setting) and θ′first secondary test setting) for the hearing device. The hearing device 2 processes the signal observed by microphone(s) of the hearing device 2 using these tuning parameters to generate audio signals y and y′, respectively, which are then presented to the user 40 for evaluation via receiver of the hearing device. The user 40 indicates preference for the first primary test setting or first secondary test setting via user input represented by r.

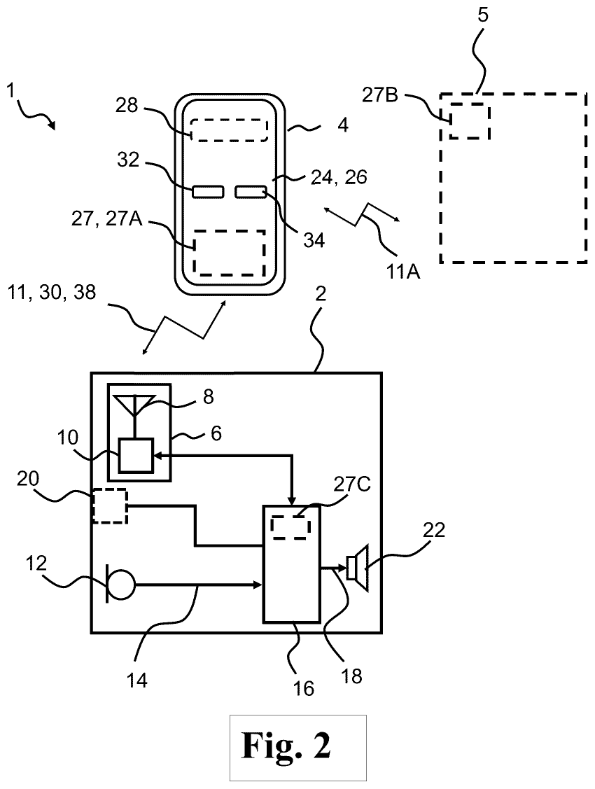

FIG. 2 is an overview of a hearing system with a fitting agent according to the present disclosure. The hearing system comprises a hearing device 2, an accessory device 4, and optionally a server device/fitting device 5. The hearing device 2 comprises a transceiver module 6 for (wireless) communication with the accessory device 4 and optionally a contralateral hearing device (not shown in FIG. 2). The transceiver module 6 comprises antenna 8 and transceiver 10 and is configured for receipt and/or transmission of wireless signals via wireless connection 11 to the accessory device 4. The accessory device 4 is configured for receipt and/or transmission of wireless signals via wireless connection 11A to the server device/fitting device 11A. The hearing device 2 comprises a set of one or a plurality of microphones comprising a first microphone 12 for provision of a first microphone input signal 14; a processor 16 for processing input signals including the first microphone input signal 14 according to one or more hearing device parameters and providing an electrical output signal 18 based on input signals; an optional user interface 20 connected to the processor 16; and a receiver 22 for converting the electrical output signal 18 to an audio output signal.

The accessory device 4 is a smartphone and comprises a user interface 24 comprising a touch display 26, a processor (not shown), and a memory (not shown).

In the hearing device system 1, the fitting agent 27 is an application installed in the memory of the accessory device 4.

The fitting agent 27 is a fitting agent for update of a user model and/or an environment model for a hearing device user and/or one or more of optimizing, determining, fitting, tuning, and modelling hearing device parameters of a hearing device.

The fitting agent 27 comprises one or more processors, such as one or more processors of accessory device and hearing device, configured to initialize a user model and an environment model, the user model comprising a plurality of user preference functions and associated user response distributions, wherein each user preference function ƒ(x; θₖ, Λₖ) is associated with a k′th environment (k=1, 2, . . . , K); obtain environment data indicative of a present environment; determine a first initial environment probability of a first environment and a second initial environment probability of a second environment using the environment and based on the environment data and the environment model, obtain a test setting comprising a primary test setting and a secondary test setting for the hearing device based on the first initial environment probability and the second initial environment probability; present the test setting to the hearing device user e.g. via wireless connection 11; obtain/detect a user input r of a preferred test setting indicative of a preference for either the primary test setting x_ref or the secondary test setting x_alt; and update the user model and/or the environment model, such as the user preference function ƒ(x; θ, Λ) and/or a user response model (user response distribution and parameter distribution), e.g. based on hearing device parameters of the preferred test setting, such as one or more, e.g. all of r, x_ref, and x_alt, and the environment data.

The fitting agent 27 implemented in the accessory device 4 is configured to update the user model and/or the environment model, such as a plurality of user preference functions of the user model, based on the primary test setting, the secondary test setting, the preferred test setting, and the environment data, such as initial environment probabilities of environments. In other words, the fitting agent is configured to update the user model based on hearing device parameters of the preferred test setting and the environment data. To update the user model may comprise updating the user preference models and the user response models based on the primary test setting, the secondary test setting, the preferred test setting, and the environment data. The fitting agent 27/accessory device 4 may be configured to transmit the primary test setting, the secondary test setting, and the preferred test setting to server device 5 that updates the user model and transmits the updated user model to the fitting agent 27/accessory device 4. Thus, fitting agent 27/accessory device 4 may be configured to receive the updated model from the server device 5. In other words, the fitting agent 27 may be distributed on accessory device 4 and one or more of hearing device 2 and server device 5. Thus, the fitting agent 27 may comprise a first part 27A implemented in accessory device 4, optional second part 27B implemented in server device, and optional third part 27C implemented in hearing device 2, such as in processor 16.

In one or more example fitting agents including fitting agent 27, 27A, 27B, 27C, to update the user model comprises to determine posteriors of parameters of the user preference functions, e.g. based on a previous parameter posteriors, such as the last or current parameter posteriors, the preferred test setting, and a non-preferred test setting. To determine a posterior of parameters of a user preference function may comprise to determine or update θₖ being the maximizing argument of the k′th ƒ and/or to determine or update Λₖ indicative of or characterizing user sensitivity to hearing device parameter changes in the k′th environment. To determine the parameter posterior optionally comprises to apply sequential estimation in the fitting agent 27, 27A, 27B, 27C. Thus, the fitting agent 27, 27A, 27B, 27C is optionally configured to determine the parameter posterior based on only the previous user model and (r, x_ref, x_alt, s).

The one or more processors of fitting agent 27 are configured to estimate a personalized environment probability of the present environment based on the updated user model, wherein the personalized environment probability is a probability of one or more user preference clusters in the domain of hearing device parameters, the one or more processors configured to update the environment model for provision of an updated environment model, e.g. based on the personalized environment probability and the environment data as also described earlier. The update of the environment model for provision of an updated environment model is performed before, in parallel to and/or after updating the user model.

In one or more example fitting agents including fitting agent 27, 27A, 27B, 27C, to obtain environment data comprises to obtain audio data and determining the environment data based on the audio data. For example, microphone 12 of the hearing device 2 may provide audio data representative of the sound received by the microphone 12, wherein the audio data can be transmitted to the accessory device 4 wherein the fitting agent 27, 27A of the accessory device 4 is configured for determining the environment data based on the audio data from the hearing device and/or wherein the fitting agent 27C of the hearing device 2 is configured for determining the environment data or at least a part of the environment data based on the audio data from microphone 12. The environment data determined by fitting agent 27C of the hearing device 2 is transmitted to the fitting agent 27A of the accessory device 4 for further processing.

In one or more example fitting agents including fitting agent 27, 27A, 27B, 27C, to obtain environment data comprises to obtain context data, e.g. from one or more sensors of the hearing device 2 and/or accessory device 4, and determining the environment data based on the context data. Determining the environment data may be based on the audio data and the context data. In one or more example fitting agents including fitting agent 27, 27A, 27B, 27C, to obtain context data comprises to obtain context data from one or more applications, such as a calendar application, of the accessory device 4.

In one or more example fitting agents including fitting agent 27, 27A, 27B, 27C, the environment data comprises a first environment probability of a first environment and/or a second environment probability of a second environment, and wherein to update the user model is based on the first probability and/or the second probability.

In one or more example fitting agents including fitting agent 27, 27A, 27B, 27C, the plurality of user preference functions comprises K user preference functions, wherein K is larger than 3, such as 5, 6, 7, 8 or more, and wherein to update the user model comprises to update posteriors of the parameters of the K user preference functions (or a subset thereof) based on the test setting, the user input of preferred test setting, and optionally one or more or the environmental data and the environment probabilities.

In one or more example fitting agents including fitting agent 27, 27A, 27B, 27C, the fitting agent is configured to obtain, such as determine one or more environment probabilities, such as an environment probability for each environment ENV_k, k=1, 2, . . . , K, and/or an environment identifier using a Gaussian mixture model and wherein to update the user model is optionally based on the environment identifier and/or the environment probabilities.

In one or more example fitting agents including fitting agent 27, 27A, 27B, 27C, the fitting agent is configured to determine whether a test criterion based on the environment data is satisfied. The fitting agent 27, 27A, 27B, 27C may be configured to, in accordance with a determination that the test criterion is satisfied, perform: obtain a test setting comprising a primary test setting and a secondary test setting for the hearing device; present the test setting to the hearing device user; obtain a user input of a preferred test setting indicative of a preference for either the primary test setting or the secondary test setting; and update the user model based on hearing device parameters of the preferred test setting and the environment data. The test criterion may be based on an uncertainty metric indicative of user preference uncertainty in the present environment.

In one or more example fitting agents including fitting agent 27, 27A, 27B, 27C, to obtain a test setting comprising a primary test setting and a secondary test setting for the hearing device comprises to determine the secondary test setting based on the environment data.

In one or more example fitting agents including fitting agent 27, 27A, 27B, 27C, the fitting agent is configured to determine and update hearing device parameters of the hearing device based on the updated user model and optionally the environment data.

In an implementation including accessory device 4, the fitting agent 27/accessory device 4 may be configured to send a control signal 30 to the hearing device 2, the control signal 30 being indicative of the primary test setting and the secondary test setting, thus enabling the hearing device 2 to output test signals accordingly.

The hearing device 2 (processor 16) is optionally configured to output a primary test signal according to the primary test setting via the receiver 22 and a secondary test signal according to the secondary test setting via the receiver 22.

The fitting agent 27, 27A, 27C (hearing device 2 (processor 16) and/or the accessory device 4) is configured to detect a user input of a preferred test setting indicative of a preference for either the primary test setting or the secondary test setting, e.g. by detecting a user input on user interface 20 or by detecting a user selection of one of a primary virtual button 32 (primary test setting is preferred) and a secondary virtual button 34 (secondary test setting is preferred) on the user interface 24 of accessory device 4.

It is to be noted that the fitting agent 27, 27A, 27B, 27C may be configured to detect a user input of a preferred test setting indicative of a preference for either the primary test setting or the secondary test setting, e.g. by receiving a wireless input signal from a secondary accessory device, such as smartwatch comprising a user interface. Thereby, a more convenient user input is provided for, which in turn increases the user friendliness of the fitting agent.

A user may initiate update of the user model and/or the environment model by pressing start button 28 on the user interface of accessory device 24. In other words, the fitting agent may detect user input indicative of start and perform the method/update of the user model in accordance with detection of the user input indicative of start.

In an implementation including accessory device 4, the accessory device 4 may be configured to send a control signal 38 to the hearing device 2, the control signal 38 being indicative of the hearing device parameters of the preferred test setting, thus enabling the hearing device to update and apply preferred hearing device parameters in the hearing device.

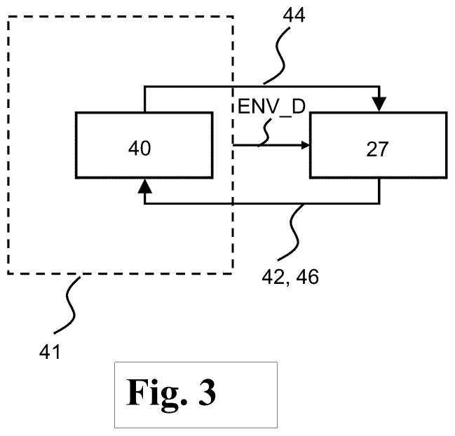

FIG. 3 illustrates interaction between a user 40 in an environment 41 and a fitting agent 27. The fitting agent 27 obtains environment data ENV_D indicative of the present environment 41, e.g. based on audio data and/or context data from one or more hearing devices worn by user 40, from user providing user input to fitting agent, from accessory device microphone, sensors, and/or applications.

For each interaction or trial indexed with index n, the fitting agent generates a primary test setting xₙ^{ref} and a secondary test setting xₙ^{alt} and presents 42 the test settings xₙ^{ref} and xₙ^{alt} for the user, e.g. by controlling hearing device for outputting primary test signal and secondary test signal respectively according to and indicative of the primary test setting and the secondary test setting. The user evaluates the two test settings xₙ^{ref} and xₙ^{alt}, and the fitting agent 27 receives and detects the user's response 44, rₙ indicative of the preferred test setting of the primary test setting and the secondary test setting. The fitting agent updates the user model based on environment data, rₙ, xₙ^{ref}, and xₙ^{alt}. The fitting agent generates n+1′th trial {xₙ₊₁^{ref}; xₙ₊₁^{alt}}, by determining the primary test setting xₙ₊₁^{ref} of the n+1′th trial based on the preferred test setting of the n′th trial and environment probabilities of the n+1′th trial, determines secondary test setting xₙ₊₁^{alt} and presents 46 the test settings xₙ₊₁^{ref} and xₙ₊₁^{alt} for the user.

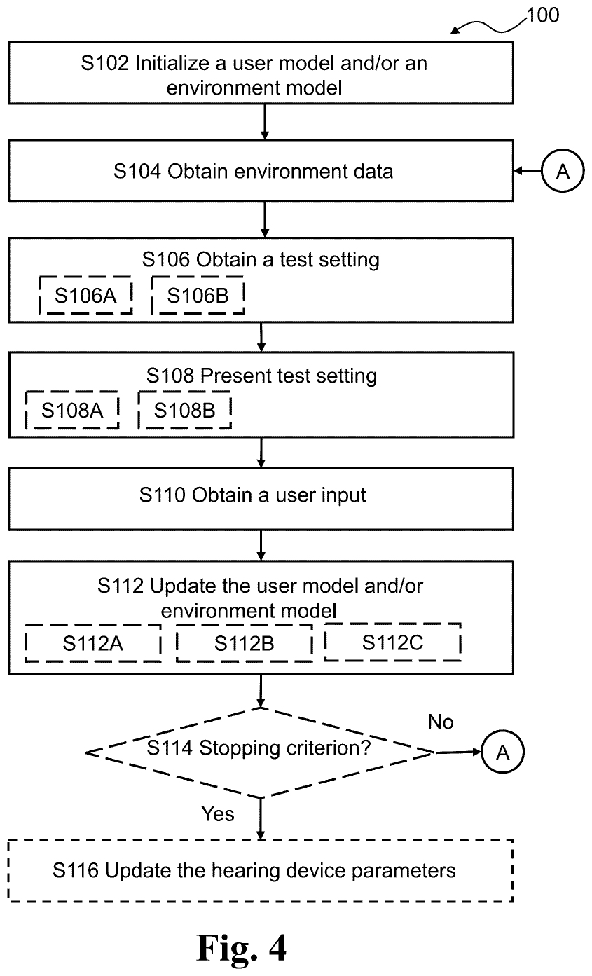

FIG. 4 is a flow diagram of an exemplary method according to the present disclosure. The method 100 is a method of updating a user model and/or an environment model for a hearing device user and/or for one or more of optimizing, determining, fitting, tuning, and modelling, such as determining hearing device parameters of a hearing device, wherein the method comprises initializing S102 an environment model and a user model, the user model comprising a plurality of user preference functions and associated user response distributions, wherein each user preference function is associated with an environment; obtaining S104 environment data indicative of a present environment; obtaining S106 a test setting comprising a primary test setting S106A and a secondary test setting S106B for the hearing device; presenting S108 the test setting to the hearing device user including outputting S108A a primary test signal in accordance with or based on the primary test setting and outputting S108B a secondary test signal in accordance with or based on the secondary test setting; obtaining S110 a user input of a preferred test setting indicative of a preference for either the primary test setting or the secondary test setting; and updating S112 the user model and/or the environment model based on hearing device parameters of the preferred test setting and non-preferred test setting and the environment data. Updating S112 the user model optionally comprises updating S112A a plurality of, such as a subset of or all of, the user preference functions of the user model. Updating S112 the user model optionally comprises updating S112B a plurality of, such as all of, the predictor/user response models of the user model.

In the method 100, updating S112 the user model and/or the environment model may comprise determining S112C posteriors of the parameters of user preference functions of the user model based on a previous or latest parameter posterior, the preferred test setting, the non-preferred test setting, and the environment data.

The method 100 optionally comprises determining S114 whether a stopping criterion is satisfied and updating S116 hearing device parameters of hearing device in accordance with the updated model, e.g. as described above in relation to the fitting agent. The method 100 optionally proceeds to S104 if it is determined that the user model has not been sufficiently updated (stopping criterion is not satisfied), i.e. further trials and user input on test settings is desired or required for an accurate and precise modelling of the environment and/or user preference functions of the user model.

The use of the terms “first”, “second”, “third” and “fourth”, “primary”, “secondary”, “tertiary” etc. does not imply any particular order, but are included to identify individual elements. Moreover, the use of the terms “first”, “second”, “third” and “fourth”, “primary”, “secondary”, “tertiary” etc. does not denote any order or importance, but rather the terms “first”, “second”, “third” and “fourth”, “primary”, “secondary”, “tertiary” etc. are used to distinguish one element from another. Note that the words “first”, “second”, “third” and “fourth”, “primary”, “secondary”, “tertiary” etc. are used here and elsewhere for labelling purposes only and are not intended to denote any specific spatial or temporal ordering.

Memory may be one or more of a buffer, a flash memory, a hard drive, a removable media, a volatile memory, a non-volatile memory, a random access memory (RAM), or other suitable device. In a typical arrangement, memory may include a non-volatile memory for long term data storage and a volatile memory that functions as system memory for the processor. Memory may exchange data with processor over a data bus. Memory may be considered a non-transitory computer readable medium.

Memory may be configured to store information in a part of the memory.

Furthermore, the labelling of a first element does not imply the presence of a second element and vice versa.

It may be appreciated that the figures comprise some modules or operations which are illustrated with a solid line and some modules or operations which are illustrated with a dashed line. The modules or operations which are comprised in a solid line are modules or operations which are comprised in the broadest example embodiment. The modules or operations which are comprised in a dashed line are example embodiments which may be comprised in, or a part of, or are further modules or operations which may be taken in addition to the modules or operations of the solid line example embodiments. It should be appreciated that these operations need not be performed in order presented. Furthermore, it should be appreciated that not all of the operations need to be performed. The exemplary operations may be performed in any order and in any combination.

It is to be noted that the word “comprising” does not necessarily exclude the presence of other elements or steps than those listed.

It is to be noted that the words “a” or “an” preceding an element do not exclude the presence of a plurality of such elements.

It should further be noted that any reference signs do not limit the scope of the claims, that the exemplary embodiments may be implemented at least in part by means of both hardware and software, and that several “means”, “units” or “devices” may be represented by the same item of hardware.

The various exemplary methods, devices, and systems described herein are described in the general context of method steps processes, which may be implemented in one aspect by a computer program product, embodied in a computer-readable medium, including computer-executable instructions, such as program code, executed by computers in networked environments. A computer-readable medium may include removable and non-removable storage devices including, but not limited to, Read Only Memory (ROM), Random Access Memory (RAM), compact discs (CDs), digital versatile discs (DVD), etc. Generally, program modules may include routines, programs, objects, components, data structures, etc. that perform specified tasks or implement specific abstract data types. Computer-executable instructions, associated data structures, and program modules represent examples of program code for executing steps of the methods disclosed herein. The particular sequence of such executable instructions or associated data structures represents examples of corresponding acts for implementing the functions described in such steps or processes.

Although features have been shown and described, it will be understood that they are not intended to limit the claimed invention, and it will be made obvious to those skilled in the art that various changes and modifications may be made without departing from the spirit and scope of the claimed invention. The specification and drawings are, accordingly to be regarded in an illustrative rather than restrictive sense. The claimed invention is intended to cover all alternatives, modifications, and equivalents.

### LIST OF REFERENCES

1 hearing device system
2 hearing device
4 accessory device
5 server device
6 transceiver module
8 antenna
10 transceiver
11 wireless connection between hearing device and accessory device
11A wireless connection between accessory device and server device
12 first microphone
14 first microphone input signal
16 processor
18 electrical output signal
20 user interface
22 receiver
24 user interface of accessory device
26 touch display
27 fitting agent
27A first part of fitting agent
27B second part of fitting agent
27C third part of fitting agent
28 (virtual) start button
30 control signal indicative of primary and secondary test setting
32 primary virtual button
34 secondary virtual button
38 control signal indicative of the hearing device parameters of the preferred test setting
40 user
41 environment
42 n′th trial, test settings xₙ^{ref} and xₙ^{alt}
44 user response to n′th trial
46 n+1′th trial, test settings xₙ₊₁^{ref} and xₙ₊₁^{alt}
50 environment model
52 preference learning system
100 Method of updating a user model and/or an environment model for a hearing device user and/or for determining hearing device parameters of a hearing device
S102 initializing a user model
S104 obtaining environment data
S106 obtaining a test setting
S106A obtaining a primary test setting
S106B obtaining a secondary test setting
S108 presenting the test setting to the hearing device user
S108A outputting a primary test signal
S108B outputting a secondary test signal
S110 obtaining a user input
S112 updating the user model and/or the environment model
S112A updating a plurality of, such as a subset of or all of, the user preference functions of the user model
S112B updating a plurality of, such as all of, the predictor/user response models of the user model
S112C determining posteriors of parameters of the user preference functions of the user model based on a previous parameter posterior, the preferred test setting, a non-preferred test setting, and the environment data
S114 determining whether stopping criterion is satisfied
S116 updating the hearing device parameters
ENV_D environment data

1. A fitting agent for a hearing device system comprising a hearing device to be worn by a hearing device user, wherein the fitting agent comprises one or more processors configured to:
initialize a user model and an environment model, the user model comprising a plurality of user preference functions and a user response distribution;
obtain environment data;
determine a first initial environment probability of a first environment and a second initial environment probability of a second environment based on the environment data and the environment model;
obtain a test setting comprising a primary test setting and a secondary test setting for the hearing device based on the first initial environment probability and the second initial environment probability;
provide the test setting for presentation to the hearing device user;
obtain a user input of a preferred test setting indicative of a preference for either the primary test setting or the secondary test setting; and
update the user model for provision of an updated user model based on the preferred test setting and the environment data.

2. The fitting agent according to claim 1, wherein the one or more processors are configured to estimate a personalized environment probability of a present environment based on the updated user model.

3. The fitting agent according to claim 2, wherein the personalized environment probability is a probability of one or more user preference clusters in a domain of hearing device parameters.

4. The fitting agent according to claim 2, wherein the one or more processors are configured to update the environment model for provision of an updated environment model based on the personalized environment probability and the environment data.

5. The fitting agent according to claim 4, wherein the one or more processors are configured to update the environment model by updating an environment distribution function based on the personalized environment probability and the environment data.

6. The fitting agent according to claim 1, wherein the environment model is a Gaussian Mixture Model.

7. The fitting agent according to claim 6, wherein the one or more processors are configured to update the environment model based on an expectation maximization algorithm.

8. The fitting agent according to claim 1, wherein the one or more processors are configured to obtain the environment data by obtaining position data indicative of a user position and determining the environment data based on the position data.

9. The fitting agent according to claim 1, wherein the one or more processors are configured to obtain the environment data by obtaining audio data indicative of sound in a present environment and determining the environment data based on the audio data.

10. The fitting agent according to claim 1, wherein the one or more processors are configured to obtain the environment data by obtaining context data indicative of a surrounding and/or an activity of the user, and determining the environment data based on the context data.

11. The fitting agent according to claim 1, wherein the one or more processors are configured to obtain a user profile, and to initialize the environment model based on the user profile.

12. The fitting agent according to claim 11, wherein the user profile comprises one or more of age, gender, hearing loss degree, and activity level; and
wherein the one or more processors are configured to initialize the environment model based on one or more of the age, the gender, the hearing loss degree, and the activity level.

13. The fitting agent according to claim 1, wherein the one or more processors are configured to obtain an environment profile, and to initialize the environment model based on the environment profile.

14. The fitting agent according to claim 1, wherein the environment data is indicative of a present environment.
